(19) United States
(12) Patent Application Publication
(10) Pub. No.: US 2024/0206514 A1
(43) Pub. Date: Jun. 27, 2024

(54) PREBIOTIC FORMULATIONS

(71) Applicant: DMK Deutsches Milchkontor GmbH, Zeven (DE)

(72) Inventors: Sven-Rainer DÖRING, Zeven (DE); Marco STEFFENS, Elsdorf (DE)

(21) Appl. No.: 18/539,597

(22) Filed: Dec. 14, 2023

(30) Foreign Application Priority Data
Dec. 15, 2022 (EP) 22213935.4

(51) Int. Cl. A23L 29/30; A23L 29/20; A23L 33/115; A23L 33/135; A23L 33/15; A23L 33/16; A23L 33/185; A23L 33/19; A23L 33/21

(52) U.S. Cl. CPC A23L 29/30; A23L 29/20; A23L 33/115; A23L 33/135; A23L 33/15; A23L 33/16; A23L 33/185; A23L 33/19; A23L 33/21

(57) ABSTRACT

What are proposed are prebiotic formulations comprising or consisting of
(a1) milk proteins and/or
(a2) plant proteins and
(b) oligosaccharides selected from the group formed by beta-galactooligosaccharides (β-GOS), alpha-galactooligosaccharides (α-GOS), fructooligosaccharides (FOS) and mannanoligosaccharides (MOS).

### FIELD OF THE INVENTION

The present invention is in the field of milk products and relates to prebiotic formulations comprising particular proteins and particular carbohydrates.

### TECHNOLOGICAL BACKGROUND

Proteins that are consumed with food supply the body with indispensable amino acids and nitrogen for the endogenous synthesis of proteins, for example structure proteins such as actin, myosin and creatin, transport proteins such as haemoglobin or transferrin, receptor proteins, immune-active proteins such as immunoglobulins, and other nitrogen compounds, for example enzymes, peptide hormones such as insulin, and DNA and RNA. Amino acids are also precursors in the synthesis of numerous metabolism products, for example gallic acids, serotonin and histamine.

In the human body, 20 different amino acids are required for synthesis of proteins; these are referred to as proteinogenic. Nine of the proteinogenic amino acids cannot be newly synthesized in the human organism; they are referred to as indispensable (formerly essential): isoleucine, leucine, lysine, methionine, phenylalanine, threonine, tryptophan, valine and, for infants, histidine. Without regular supply of these indispensable amino acids, deficiency symptoms can occur.

The most important protein sources have long been fish and meat. In the last few years, however, there has been an increasing trend towards meat-free nutrition—whether for ethical or health reasons—the effect of which has been that proteins from alternative sources have become more important. Particular mention should be made here of milk proteins and, as of recently, plant proteins as well, especially since these are classified as vegan. A disadvantage, however, is that the non-animal proteins, especially plant proteins, have a bitter and more or less pronounced characteristic taste that has to be masked.

At the same time, there is a need on the market for protein products having further functionalities, especially in the athlete nutrition sector.

### RELEVANT PRIOR ART

EP 2124639 B1 (FRAUNHOFER) relates to a method of modifying the taste profile of a plant protein formulation, especially a protein formulation from a pulse. The protein preparation is contacted with water-soluble carbohydrates in an aqueous solution before being added to food, wherein the contact has an advantageous effect on the taste profile of protein preparations from legumes, such that the preparations can be used in foods without significantly altering the taste thereof.

EP 0936875 B1 (NOVOZYMES) relates to the production of a flavouring for foods. In particular, the invention provides a process for producing a flavouring for foods, comprising the steps of producing an aqueous slurry of plant protein and plant carbohydrate, treating the slurry with a protease, treating the slurry with a carbohydrase, and maturing. This method can induce various taste properties in the protein hydrolysate.

### OBJECT OF THE INVENTION

It was therefore an object of the present invention to satisfy the two needs on the market, namely firstly for non-animal protein formulations having improved taste properties and secondly for protein formulations having additional functionalities, simultaneously with a new product. The formulations were also to be sufficiently flexible that they meet the wishes of different groups of consumers, i.e. can be classified as vegetarian, purely milk-based or vegan.

### DESCRIPTION OF THE INVENTION

The invention firstly provides prebiotic formulations comprising or consisting of
(a1) milk proteins and/or
(a2) plant proteins and
(b) oligosaccharides selected from the group formed by beta-galactooligosaccharides (β-GOS), alpha-galactooligosaccharides (α-GOS), fructooligosaccharides (FOS) and mannanoligosaccharides (MOS).

It has been found that, surprisingly, oligosaccharides of the type mentioned are an excellent supplement to protein components because they firstly make them prebiotic and secondly increase the nutritional value without having to add sugar to the products. The oligosaccharides impart texture/structure and a certain sweetness to the formulations, and additionally mask the sometimes bitter characteristic taste of the proteins. In this connection, it should be pointed out that the masking of a flavour must not be confused with simply covering up the flavour by the amount of another substance. Masking means that two flavour directions combine and give rise to a new taste; it is typical here that the masking requires significantly smaller amounts compared to covering-up.

The invention additionally provides the desired building block system in which any desired combination from two product groups—proteins and oligosaccharides—and any two members is possible. A combination of milk proteins and β-GOS that have been obtained by transgalactosylation of lactose is, for example, fully milk-based. A formulation containing plant proteins and α-GOS based on sucrose, by contrast, would be vegan.

### Milk Proteins

Milk proteins consist of the components casein (about 80%) and whey protein (about 20%). Casein in milk is colloidally distributed in phosphorylated form and associated with calcium (calcium-proteinate-phosphate particles). It contains all essential amino acids that are not destroyed even in the processing of the milk. The separation of casein affords α-, β- and γ-caseins, which can be divided in turn into further fractions. An important subfraction is κ-casein, which exerts the function of a protective colloid in milk.

In the production of milk proteins, the two protein components are not separated. Instead, cow's milk is taken as the base and the protein is filtered out. A milk protein concentrate is obtained. The milk protein concentrate lacks a majority of the fat still present in the milk powder and of the lactose. If these two nutrients are removed from the concentrate, all that remains is the protein concentrate, called the milk protein isolate.

In the context of the present invention, the milk proteins may be selected from the group formed by casein, milk protein concentrates, milk protein isolates, whey proteins, whey protein concentrates and protein-containing milk powders.

### Plant Proteins

Plant proteins in the context of the invention (component a) can be obtained from potatoes, soya, peas, lupins, oilseed rape or other high-protein fruits, for example sunflower or pumpkin seeds or the like. The harvested protein-containing fruits are mechanically comminuted and defatted. The result is flakes or a protein-rich powder. Subsequently, a protein concentrate is obtained using solvents, which is optionally purified and concentrated further to give protein isolate. The flakes or meal are admixed with water and converted to a slurry. The low-protein fibres and solids are separated from the protein-rich solution in the next step with the aid of industrial centrifuges. This is followed by what is called the precipitation. The pH of the protein-rich solution is adjusted here to the isoelectric point. This results in sedimentation of the protein particles. These are then separated in turn from the liquor by means of centrifuges. In order to remove all constituents of the mother liquor from the precipitated and removed protein, the protein is admixed again with water and separated off again with the aid of centrifugal force. In the case of dry extrusion, with supply of heat, pressure and auxiliaries, an intermediate with a low water content is produced. These likewise suitable proteins are referred to as TVP (texturized vegetable protein) and have a dry consistency in the form of grains, strips or flakes. In the case of wet extrusion, alternatively, operation is effected with a higher water content. The moisture content of the intermediate is therefore closer to the water content of the end product. The intermediate is referred to as HMMA (high moisturized meat analogues).

### Oligosaccharides

Beta-galactooligosaccharides (β-GOS) (component b1)

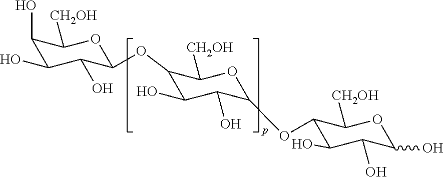

also known as oligogalactosyllactoses, oligogalactoses, oligolactoses or transgalactooligosaccharides (TOS), belong to the group of the prebiotics. GOS is present in commercial products such as food for infants and adults. Because of the configuration of their glycosidic bonds, galactooligosaccharides (GOS) largely withstand hydrolysis by saliva enzymes and intestinal digestion enzymes. Galactooligosaccharides are therefore classified as prebiotics, defined as indigestible food constituents that have a beneficial effect on the hosts in that they stimulate the growth and/or activity of useful bacteria in the large intestine. The elevated activity of these health-promoting bacteria leads to a number of effects, both directly via the bacteria themselves or indirectly via the organic acids that they produce via fermentation. Examples of effects are the stimulation of immune functions, the uptake of essential nutrients, and the synthesis of particular vitamins. The typical obtaining of GOS comprises the following steps:
1. concentration of a lactose solution (lactose, acid whey, milk permeate) with the aim of achieving a dry mass of at least 30% by weight;
2. UHT treatment for sterilization at 85-140 C for 5 to 300 s;
3. addition of a beta-galactosidase (for example from Aspergillus Oryzae or Bacillus circulans) and establishment of the optimal pH and temperature conditions for the enzyme (e.g. pH=4.5, 55° C.);
4. a dwell time of generally more than 30 to not more than 120 min (depending on the enzyme), since there is otherwise redissociation;
5. inactivation of the enzyme, for example by high-temperature pasteurization (90° C., 10 min) or a pH shift;
6. purification, optionally concentration, spray drying.

alpha-Galactooligosaccharides (α-GOS) (component b2) alpha-Galactooligosaccharides can be produced by transgalactosylation of alpha-galactosides (for example melibiose) by the enzyme alpha-galactosidase. alpha-GOS may contain up to 6 galactose monomers and have a terminal glucose molecule. These are alpha-(1,6)-bonded to one another. They have similar prebiotic properties to beta-galactooligosaccharides.

Fructooligosaccharides (FOS) (component b3)

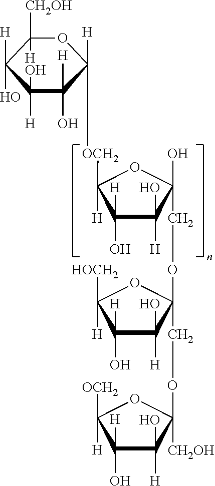

FOS are polysugars that may contain up to 10 monomers. Fructooligosaccharides can be obtained from sucrose by transfructosylation by means of a beta-fructosidase. As well as a beta-1,2 linkage, they also have other means of bonding of the monomeric sugars. FOS can likewise be produced by the enzymatic or acid-catalysed hydrolysis of high-polymerized inulin. Since these glycosidic bonds cannot be split by salivary enzymes and digestive enzymes, the result is a prebiotic effect.

Mannanoligosaccharides (MOS) (component b4)

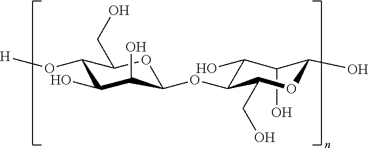

MOS are mannanoligosaccharides and occur naturally as constituents of cell walls in yeasts. They also occur as mannose-protein complexes. MOS products are purified yeast cell wall products from different yeast strains.

Both for reasons of nutrition and physiology and with regard to optimal masking of the bitter taste note of the proteins, oligosaccharides having a high DPE (“degree of polymerization”) are preferred, especially with a DP in the range from about 10 to about 80, preferably about 30 to about 70 and especially about to about 60. Such oligosaccharides are obtained, for example, when the transgalactosylation is conducted in two stages with two different enzymes.

### Formulations

The prebiotic formulations of the invention may further comprise auxiliaries and additives selected from the group formed by animal and/or vegetable fats, starch products, dietary fibres, thickeners, flavourings, further prebiotics, probiotics, food acids, salts and dyes, and mixtures thereof.

Animal and/or Vegetable Fats

Fats (component c1) are lipids that contain essentially saturated fatty acids and are therefore in solid form at ambient temperature. Solid animal fats include bovine tallow, pork lard and milk fat. Examples of vegetable fats are coconut fat, palm fat, palm kernel fat and shea butter.

### Starch Products

In the context of the present invention, the term “starch products” (component c2) is formed by natural starches, and likewise by chemically or enzymatically modified starches, provided that these are approved for human nutrition, and dextrins.

Potato starch Potatoes contain about 75% water, 21% starch and 4% other substances. For production of potato starch, they are traditionally comminuted to maximum fineness by sawtooth cylinders rotating at high speed with supply of water. This is followed by leaching of the pulp, in which the cells have been ruptured as completely as possible, i.e. the starch grains should be exposed, from a metal screen on which brushes rotate slowly, with water. In the case of larger-scale operations, continuous apparatuses are used, in which the pulp is transported by a chain gradually across a long, inclined screen while being leached, and the water flowing onto the already almost exhausted pulp, which thus takes up only a very small amount of ground starch, is also directed onto the fresh pulp. The leached pulp contains 80-95% water, but still about 60% starch in the dry matter, and serves as animal feed, and also for production of starch sugar, production of spirits and papermaking; the wash water has been used for irrigation of fields, but it has also been possible to utilize the nitrogen-containing constituents of the potato tuber water as animal feed. Since the pulp still contains a very large amount of starch, it is comminuted between rollers in order to open all the cells, and leached once again. In another method, the potatoes are cut into slices, freed of their juice by maceration in water, and layered together with brushwood or wattles to give heaps in which they rot completely at a temperature of 30-40° C. within about eight days and are transformed to a loose, pulp-like mass from which the starch can be leached readily. The water flowing away from the sieves contains the juice constituents of the potatoes in dissolved form, and starch and fine fibres that have passed through the screen in suspended form. This water is stirred up in vats, left to stand for a short time in order that sand and small stones can fall to the base, allowed to flow through a fine screen in order to retain coarser fibres, and then introduced into a vat in which the starch, and on it the fibres, are separated out. The upper layer of the sediment is therefore removed after the water has been drained and used directly as starch sludge, or purified further by leaching it with a large amount of water on an agitated screen made of fine silk gauze, through the meshes of which the starch will pass, but not the fibres. The main mass of the starch is repeatedly stirred up with pure water in the vat and freed of the upper impure starch after each settling step. It is also possible to allow the crude starch to flow with water through a very slightly inclined channel, in the upper part of which the heavy pure starch is deposited, while the lighter fibres are carried onwards by the water.

Centrifugal machines are often also used, in which the heavy starch is first deposited on the vertical wall of the fast-rotating screen drum, while the light fibre still remains suspended in the water. But the water escapes through the screen wall, and the starch can ultimately be lifted out of the centrifugal machine in solid blocks, the inner layer of which is formed by the fibre. The moist (green) starch containing about 33-45% water is processed directly to glucose, but for all other purposes it is dewatered on filter presses or on sheets of calcined gypsum that soak up plenty of water, also using an air pump, and dried at a temperature below 60° C. It is traded in lumps or, having been crushed between rolls and sieved, as ground material. The moist starch is sometimes kneaded with a little gluten and driven through a perforated iron plate, and then the strips obtained are dried on wattles. In order to conceal a yellowish hue of the starch, a little ultramarine is added thereto before the last wash.

Wheat starch Wheat starch is produced from white, thin-husked, farinaceous wheat. This contains about 58-64% starch, and also about 10% gluten and 3-4% cellulose, which mainly forms the husks of the grain. The properties of the gluten determine the variances in the wheat starch fabrication from the obtaining of the starch from potatoes. In the traditional Halle process or acid process, the wheat is softened in water, squashed between rolls and left to ferment under water; fermentation is initiated by acid water from an earlier process and affords acetic acid and lactic acid in which the gluten is dissolved or at least loses its viscosity such that it is possible to separate out the starch after 10-20 days in a wash drum with a sieve-like pattern of holes. The water flowing out of the drum firstly deposits starch in a vat, then an intimate mixture of starch with gluten and husk particles (size, starch sludge), and lastly a sludgy mass consisting predominantly of gluten. This raw starch is purified and then dried similarly to potato starch, breaking down into powder, or, if it still contains small amounts of gluten, giving what is called crystal starch, which is incorrectly assumed by normal consumers to be particularly pure.

In the traditional Alsace method, the swollen wheat is squeezed by upright millstones with a strong inflow of water and extracted by washing immediately. The effluxing water, as well as starch, contains a large amount of gluten and husked particles, and is either left to ferment and then processed further as in the preceding process, or transferred directly to centrifugal machines where a large amount of gluten is separated out and a crude starch is obtained, which is purified further by fermentation etc. The residues obtained in this process have considerably higher agricultural value than those formed in the Halle process. But if the intention is to utilize the gluten even more advantageously, a firm, tough dough is made from wheat flour and, after about one hour, processed into 1 kg pieces in a channel-shaped trough with supply of water using a slightly fluted roll. In the course of this, the starch is washed out of the gluten and flows away with the water, while the gluten is retained as a viscous, threading material.

Rice starch Rice contains about 70-75% starch, as well as 7-9% insoluble protein-like substances, but these are dissolved for the most part by softening the rice in quite weak sodium hydroxide solution. The rice is then comminuted in a mill with constant inflow of weak alkali, the slurry is subjected to sustained treatment with alkali and water in a vat, and is left to settle for a short time, in order that coarser particles fall to the base, and the water in which pure starch is suspended is drawn off. The starch is washed out of the sediment by water in a rotating screen cylinder, and then it is freed of the gluten by treatment with alkali and slurrying. The purer starch obtained first can then be left to settle out, the upper, impure layer is removed, the rest is treated in the centrifugal machine, and the pure starch is dried.

Maize starch Maize is softened four to five times for 24 hours each time in water at 35° C., washed and then passed through two milling operations. The flour falls into a water-filled vat with paddle stirrers, and passes therefrom onto silk fabric that retains only the coarse bran. The starch-laden water that has passed through the fabric goes into troughs, then passes through two fine fabrics and finally onto a slightly inclined slate tablet of 80-100 m in length, on which the starch is deposited. The water flowing away, which contains only traces of starch, is left to stand, the sediment is pressed to cakes, in order to use it as animal feed. Particular preference is given to the use of rice starch or maize starch.

Dextrins Dextrins or maltodextrins are starch degradation products which, in terms of their molecular size, are between oligosaccharides and starch. They typically occur in the form of white or pale yellow powder. They are obtained mainly from wheat starch, potato starch, tapioca starch and maize starch by dry heating (>150° C.) or under the action of acid. In nature, dextrin is produced, for example, by Bacterium macerans. Dextrins are also formed by the enzymatic degradation of starch by amylase. Preference is given to dextrins with 5 to 20 and especially 6 to 10 dextrose equivalents (DE units).

### Dietary Fibres

Dietary fibres (component c3) are largely indigestible food constituents, usually carbohydrates, that are usually present in plant-based foods and are an important part of human nutrition. In yet a further preferred embodiment of the present invention, the dietary fibre is selected from the group consisting of inulin, carob seed flour, pectin, dextrin, maltodextrin, cellulose, lignin and alginate, or mixtures thereof.

### Emulsifiers

Emulsifiers (component c4) are notable for the important property of being soluble both in water and in fat. Emulsifiers usually consist of a fat-soluble and a water-soluble component. They are used whenever water and oil are to be converted to a stable, homogeneous mixture. Suitable emulsifiers that are used in the food-processing industry are selected from: ascorbyl palmitate (E 304) lecithin (E 322) phosphoric acid (E 338) sodium phosphate (E 339) potassium phosphate (E 340) calcium phosphate (E 341) magnesium orthophosphate (E 343) propylene glycol alginate (E 405) polyoxyethylene(8) stearate (E 430) polyoxyethylene stearate (E 431) ammonium phosphatides (E 442) sodium phosphate and potassium phosphate (E 450) sodium salts of food fatty acids (E 470 a) mono- and diglycerides of food fatty acids (E 471) acetic acid monoglycerides (E 472 a) lactic acid monoglycerides (E 472 b) citric acid monoglycerides (E 472 c) tartaric acid monoglycerides (E 472 d) diacetyltartaric acid monoglycerides (E 472 e) sugar esters of food fatty acids (E 473) sugar glycerides (E 474) polyglycerides of food fatty acids (E 475) polyglycerol polyricinoleate (E 476) propylene glycol esters of food fatty acids (E 477) sodium stearoyllactylate (E 481) calcium stearoyl-2-lactylate (E 482) stearyl tartrate (E 483) sorbitan monostearate (E 491) stearic acid (E 570).

### Thickeners

Thickeners (component c5) are substances capable primarily of binding water. Removal of unbound water results in an increase in viscosity. Over and above a concentration characteristic of each thickener, network effects also occur as well as this effect, which lead to a usually greater-than-proportional increase in viscosity. In this case, it is said that molecules ‘communicate’ with one another, i.e. become interlooped. Most thickeners are linear or branched macromolecules (e.g. polysaccharides or proteins) that can interact with one another via intermolecular interactions, such as hydrogen bonds, hydrophobic interactions or ionic relationships. Extreme cases of thickeners are sheet silicates (bentonites, hectorites) or hydrated SiO₂ particles that are dispersed as particles and can bind water in their solid-state structure or can interact with one another on account of the interactions described.

Useful thickeners are conventionally gelatins in particular. Since animal-based products are now undesirable in many cases, it is possible to use as an alternative plant-based products, for example
E 400—alginic acid
E 401—sodium alginate
E 402—potassium alginate
E 403—ammonium alginate
E 404—calcium alginate
E 405—propylene glycol alginate
E 406—agar agar
E 407—carrageenan, furcelleran
E 407—carob seed flour
E 412—guar seed flour
E 413—tragacanth
E 414—gum arabic
E 415—xanthan gum
E 416—karaya (Indian tragacanth)
E 417—tara bean flour (Peruvian carob seed flour)
E 418—gellan gum
E 440—pectin, Opekta
E 440ii—amidated pectin
E 460—microcrystalline cellulose, cellulose powder
E 461—methylcellulose
E 462—ethylcellulose
E 463—hydroxypropylcellulose
E 465—methylethylcellulose
E 466—carboxymethylcellulose, sodium carboxymethylcellulose

### Flavourings

The formulations according to the invention may comprise one or more flavourings (component c6). Typical examples include: acetophenone, allyl caproate, alpha-ionone, beta-ionone, anisaldehyde, anisyl acetate, anisyl formate, benzaldehyde, benzothiazole, benzyl acetate, benzyl alcohol, benzyl benzoate, beta-ionone, butyl butyrate, butyl caproate, butylidenephthalide, carvone, camphene, caryophyllene, cineol, cinnamyl acetate, citral, citronellol, citronellal, citronellyl acetate, cyclohexyl acetate, cymol, damascone, decalactone, dihydrocoumarin, dimethyl anthranilate, dodecalactone, ethoxyethyl acetate, ethylbutyric acid, ethyl butyrate, ethyl caprinate, ethyl caproate, ethyl crotonate, ethyl furaneol, ethyl guaiacol, ethyl isobutyrate, ethyl isovalerate, ethyl lactate, ethyl methylbutyrate, ethyl propionate, eucalyptol, eugenol, ethyl heptylate, 4-(p-hydroxyphenyl)-2-butanone, gamma-decalactone, geraniol, geranyl acetate, grapefruit aldehyde, methyl dihydrojasmonate (e.g. Hedion®), heliotropine, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxybenzylacetone, isoamylalcohol, isoamyl isovalerate, isobutyl butyrate, isobutyraldehyde, isoeugenol methyl ether, isopropylmethylthiazole, lauric acid, levulinic acid, linalool, linalool oxide, linalyl acetate, menthol, menthofuran, methyl anthranilate, methylbutanol, methylbutyric acid, 2-methylbutyl acetate, methyl caproate, methyl cinnamate, 5-methylfurfural, 3,2,2-methylcyclopentenolone, 6,5,2-methylheptenone, methyl dihydrojasmonate, methyl jasmonate, 2-methyl methylbutyrate, 2-methyl-2-pentenolic acid, methyl thiobutyrate, 3,1-methylthiohexanol, 3-methylthiohexyl acetate, nerol, neryl acetate, trans,trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinols, 8,3-thiomenthanone, 4,4,2-thiomethylpentanone, thymol, delta-undecalactone, gamma-undecalactone, valencene, valeric acid, vanillin, acetoin, ethylvanillin, ethylvanillin isobutyrate (=3-ethoxy-4-isobutyryloxybenzaldehyde), 2,5-dimethyl-4-hydroxy-3(2H)-furanone and derivatives thereof (here preferably homofuraneol) (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (here preferably ethyl maltol), coumarin and coumarin derivatives, gamma-lactones (here preferably gamma-undecalactone, gamma-nonalactone, gamma-decalactone), delta-lactones (here preferably 4-methyl deltadecalactone, massoia lactone, deltadecalactone, tuberolactone), methyl sorbate, divanillin, 4-hydroxy-2(or 5)-ethyl-5(or 2)-methyl-3(2H)-furanone, 2-hydroxy-3-methyl-2-cyclopentenone, 3-hydroxy-4,5-dimethyl-2(5H)-furanone, acetic acid isoamyl ester, butyric acid ethyl ester, butyric acid n-butyl ester, butyric acid isoamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenylglycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanthiol, bis(2-methyl-3-furyl) disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamaldehyde, cinnamyl alcohol, methyl salicylate, isopulegol and (not explicitly stated here) stereoisomers, enantiomers, positional isomers, diastereomers, cis/trans isomers or epimers of these substances.

The flavourings may also be added in solid or pasty form, for example as dried seasoning mixtures or chopped herbs.

### Probiotic Microorganisms

Probiotic microorganisms, which are also referred to as “probiotics” (component c7), are living microorganisms that have properties useful to the host. According to the FAO/WHO definition, they are “live microorganisms which when administered in adequate amounts confer a health benefit on the host”. Lactic acid bacteria (LAB) and bifidobacteria are the best-known probiotics, but it is also possible to use various yeasts and bacilli. Probiotics are typically consumed as a constituent of fermented foods, to which special living cultures have been added, for example yoghurt, soya yoghurt or other probiotic foods. In addition, tablets, capsules, powders and sachets that contain these microorganisms in freeze-dried form are also obtainable. Table A gives an overview of commercial probiotics and the corresponding health claims that can be used as component (b1) in the context of the present invention.

TABLE A
Probiotic microorganisms

| Strain | Name | Manufacturer | Claim |
|---|---|---|---|
| Bacillus coagulans GBI- | GanedenBC | Ganeden | Increases the immune |
| 30, 6086 | | Biotech | response on viral infection |
| Bifidobacterium animalis | Probio-Tec | Chr. Hansen | Human clinical studies have |
| subsp. lactis BB-12 | Bifidobacterium BB-12 | | shown that BB-12 alone or in |
| | | | combination has a positive |
| | | | effect on the gastrointestinal |
| | | | system. |
| Bifidobacterium infantis | Align | Procter & | In a preliminary study, it was |
| 35624 | | Gamble | shown that the bacterium can |
| | | | reduce abdominal pain. |
| Lactobacillus acidophilus | | Danisco | A study shows that the side |
| NCFM | | | effects of antibiotic treatments |
| | | | are reduced. |
| Lactobacillus paracasei | | | |
| St11 (or NCC2461) | | | |
| Lactobacillus johnsonii | | Nestlé | Reduces gastritis complaints |
| La1 (=Lactobacillus | | | and reduces inflammation. |
| LC1, Lactobacillus | | | |
| johnsonii NCC533) | | | |
| Lactobacillus plantarum | GoodBelly/ProViva/ProbiMage | Probi | May improve IBS symptoms; |
| 299v | | | but more studies required. |
| Lactobacillus reuteri | | BioGaia | First signs of efficacy against |
| American Type Culture | | | gingivitis, fever in children |
| Collection\|ATTC 55730 | | | and reduction in days of |
| (Lactobacillus reuteri | | | illness in adults. |
| SD2112) | | | |
| Lactobacillus reuteri | | | |
| Protectis (DSM 17938, | | | |
| daughter strain of ATCC | | | |
| 55730) | | | |
| Saccharomyces boulardii | DiarSafe and others | Wren | Limited evidence in the |
| | | Laboratories | treatment of acute diarrhoea. |
| Lactobacillus rhamnosus | Bion Flore Intime/Jarrow | Chr. Hansen | Evidence of efficacy against |
| GR-1 & Lactobacillus | Fem-Dophilus | | vaginitis in one study. |
| reuteri RC-14 | | | |
| Lactobacillus acidophilus | Florajen3 | American | First signs of efficacy against |
| NCFM & Bifidobacterium | | Lifeline, Inc | CDAD |
| bifidum BB-12 | | | |
| Lactobacillus acidophilus | Bio-K + CL1285 | Bio-K + | Pointers of improvement in |
| CL1285 & Lactobacillus | | International | digestion, especially with |
| casei LBC80R | | | regard to lactose intolerance. |
| Lactobacillus plantarum | Bravo Friscus/ProbiFrisk | Probi | Studies currently underway with |
| HEAL 9 & Lactobacillus | | | regard to efficacy against colds. |
| paracasei 8700:2 | | | |

Two further forms of lactic acid bacteria, namely Lactobacillus bulgaricus and Streptococcus thermophilus, are likewise suitable as probiotics. Specific fermented products based on such lactic acid bacteria are also usable; for example mixed pickles, fermented soybean paste, for example tempeh, miso and doenjang; kefir; buttermilk, kimchi; pao cai; soy sauce or zha cai.

### Prebiotic Substances

In a further configuration of the invention, the formulations may comprise additional prebiotic substances (“prebiotics”) (component c8). Prebiotics are defined as indigestible food constituents, the administration of which stimulates growth or activity of a number of useful bacteria in the large intestine. The addition of prebiotic compounds improves the stability of the anthocyanins against degradation processes in the gastrointestinal tract. Various substances, especially carbohydrates that are particularly preferred as prebiotics in the context of the invention, are specified hereinafter, namely in particular
fructooligosaccharides
inulins
isomaltooligosaccharides
lactilol
lactulose
pyrodextrins
soya oligosaccharides
xylooligosaccharides and
specific biopolymers

Fructooligosaccharides, or FOS for short, especially include short-chain representatives having 3 to 5 carbon atoms, for example D-fructose and D-glucose. FOS, also called neosugars, are produced commercially on the basis of sucrose and the fructosyltransferase enzyme which is obtained from fungi. FOS especially promote the growth of bifidobacteria in the intestine and are marketed in the USA particularly together with probiotic bacteria in various functionalized foods.

Inulins belong to a group of naturally occurring fructose-containing oligosaccharides. They belong to a class of carbohydrates that are referred to as fructans. They are obtained from the roots of the chicory plant (Cichorium intybus) or what are called Jerusalem artichokes. Inulins consist predominantly of fructose units and typically have a glucose unit as end group. The fructose units are linked to one another via a beta-(2-1)-glycosidic bond. The average degree of polymerization of inulins that are employed as prebiotics in the foods sector is 10 to 12. Inulins likewise stimulate the growth of bifidobacteria in the large intestine.

Isomaltooligosaccharides are a mixture of alpha-D-linked glucose oligomers, including isomaltose, panose, isomaltotetraose, isomaltopentaose, nigerose, kojibiose, isopanose and higher branched oligosaccharides. Isomaltooligosaccharides are produced via various enzymatic routes. They likewise stimulate the growth of bifidobacteria and lactobacilli in the large intestine. Isomaltooligosaccharides are used especially in Japan as food additives in functionalized foods. They are now also widespread in the USA.

Lactilol is the disaccharide of lactulose. It is used in medicine to counter constipation and in the case of hepatic encephalopathy. In Japan, lactilol is used as a prebiotic. It withstands degradation in the upper digestive tract, but is fermented by various intestinal bacteria, which leads to a rise in the biomass of bifidobacteria and lactobacilli in the intestine. Lactilol is also known by the chemical name 4-O-(beta-D-galactopyranosyl)-D-glucitol. The medical field of use of lactitol in the USA is limited for lack of studies; in Europe it is preferably used as a sweetener.

Lactosucrose is a trisaccharide which is formed from D-galactose, D-glucose and D-fructose. Lactosucrose is produced by enzymatic transfer of the galactosyl residue in lactose to sucrose. It is not degraded in the stomach or in the upper part of the gastrointestinal tract, and is consumed exclusively by bifidobacteria for growth. From a physiological standpoint, lactosucrose acts as a simulator for the growth of intestinal flora. Lactosucrose is likewise known as 4G-beta-D-galactosucrose. It is widely used in Japan as a food additive and as a constituent of functionalized foods, especially also as an additive for yoghurts. Lactosucrose is currently also being tested in the USA for a similar end use.

Lactulose is a semisynthetic disaccharide formed from D-lactose and D-fructose. The sugars are linked by a beta-glycosidic bond, which makes them resistant to hydrolysis by digestive enzymes. Instead, lactulose is fermented by a limited number of intestinal bacteria, which leads to growth of lactobacilli and bifidobacteria in particular. Lactulose in the USA is a prescribed medicament to counter constipation and hepatic encephalopathy. In Japan, by contrast, it is freely sold as a food additive and constituent of functionalized foods.

Pyrodextrins comprise a mixture of glucose-containing oligosaccharides that are formed on hydrolysis of starch. Pyrodextrins promote the proliferation of bifidobacteria in the large intestine. They are also not degraded in the upper region of the intestine.

Soya oligosaccharides are specific saccharides that are found essentially only in soybeans and additionally in other beans and peas. The two crucial representatives are the trisaccharide raffinose and the tetrasaccharide stachyose. Raffinose is composed of one molecule each of D-galactose, D-glucose and D-fructose. Stachyose consists of two molecules of D-galactose and one molecule each of D-glucose and D-fructose. Soya oligosaccharides stimulate the growth of bifidobacteria in the large intestine and are already being used in Japan as food additives and in functionalized foods. In the USA, they are currently being tested for this use.

Xylooligosaccharides contain beta-1,4-linked xylose units. The degree of polymerization of the xylooligosaccharides is between 2 and 4. They are obtained by enzymatic hydrolysis of the polysaccharide xylan. They are already being marketed as food additives in Japan; they are still in the test phase in the USA.

Suitable biopolymers that are likewise useful as prebiotics, for example beta-glucans, are notable in that they are produced on a plant basis; for example, useful raw material sources include cereals such as oats and barley, but also fungi, yeasts and bacteria. Microbially produced cell wall suspensions or whole cells with a high beta-glucan content are also suitable. Residual contents of monomers have 1-3 and 1-4 or 1-3 and 1-6 linkages, and the content may vary significantly. What are preferably obtained are beta-glucans based on yeasts, especially Saccharomyces, specifically Saccharomyces cerevisiae. Other suitable biopolymers are chitin and chitin derivatives, especially oligoglucosamine and chitosan, which is a typical hydrocolloid.

### Food Acids

The term “food acids” (component c9) refers to organic acids, fruit acids or phosphoric acids, which, because of their taste and other properties advantageous for food purposes, are used as additives, especially as food acids or acid regulators in food production, for example:
E 260—acetic acid
E 270—lactic acid
E 290—carbon dioxide
E 296—malic acid
E 297—fumaric acid
E 330—citric acid
E 331—sodium citrate
E 332—potassium citrate
E 333—calcium citrate
E 334—tartaric acid
E 335—sodium tartrate
E 336—potassium tartrate
E 337—sodium potassium tartrate
E 338—phosphoric acid
E 353—metatartaric acid
E 354—calcium tartrate
E 355—adipic acid
E 363—succinic acid
E 380—triammonium citrate
E 513—sulfuric acid
E 574—gluconic acid
E 575—glucono-delta-lactone

Preferably, in the context of the present invention, the food acids are selected from the group consisting of lactic acid, tartaric acid, acetic acid, malic acid, citric acid and fumaric acid. Even though a food product according to the invention may contain food acids that may (also) be used because of their taste in the context of the present invention, the food acids in the context of the present text are not assigned the term “flavouring” within the context of the wording of the claim (see above for flavourings to be used with preference). Such (further) flavourings are thus always additionally present.

### Sweeteners

The formulations may additionally comprise sweeteners (component c10). Since these are low-calorie or at least reduced-calorie foods, it is possible to use conventional sugars for sweetening, albeit less preferred. Instead, preference is given to using plant-based alternatives.

Useful sweeteners or sweet-tasting additives include firstly carbohydrates and specifically sugars, for instance sucrose, trehalose, lactose, maltose, melicitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, or maltodextrin. Likewise suitable are plant-based formulations that contain these substances, for example based on sugarbeet (Beta vulgaris ssp., sugar fractions, sugar syrup, molasses), sugarcane (Saccharum officinarum ssp., molasses, sugarcane syrup), maple syrup (Acer ssp.) or agaves (agave syrup).

Also useful are
synthetic, i.e. generally enzymatically produced, starch or sugar hydrolysates (invert sugar, fructose syrup),
fruit concentrates (for example based on apples or pears);
sugar alcohols (e.g. erythritol, threitol, arabitol, ribotol, xylitol, sorbitol, mannitol, dulcitol, lactitol);
proteins (e.g. miraculin, monellin, thaumatin, curculin, brazzein);
sweeteners (e.g. magap, sodium cyclamate, acesulfame K, neohesperidine dihydrochalcone, saccharin sodium salt, aspartame, superaspartame, neotame, alitame, sucralose, stevioside, rebaudioside, especially A and D, lugduname, carrelame, sucrononate, sucrooctate, monatin, phenylodulcin);
sweet-tasting amino acids (e.g. glycine, D-leucine, D-threonine, D-asparagine, D-phenylalanine, D-tryptophan, L-proline);
further sweet-tasting low molecular weight substances, for example hernandulcin, dihydrochalcone glycosides, glycyrrhizin, glycerrhetic acid, derivatives and salts thereof, liquorice extracts (Glycyrrhizza glabra ssp.), Lippia dulcis extracts, Momordica ssp. extracts or
single substances, for example Momordica grosvenori [Luo Han Guo] and the mogrosides obtained therefrom, Hydrangea dulcis or Stevia ssp. (e.g. Stevia rebaudiana), and Rubus extracts.

### Antioxidants and Vitamins

In a further embodiment of the present invention, the formulations may comprise antioxidants and/or vitamins (component c11).

In the food industry, both natural and synthetic antioxidants are used. Natural and synthetic antioxidants differ primarily in that the former are naturally occurring in food and the latter are produced synthetically. For instance, natural antioxidants, if they are to be used as food additive, are obtained from vegetable oils, for example. Vitamin E—also referred to as tocopherol—is frequently produced from soya oil, for example. Synthetic antioxidants, such as propyl gallate, octyl gallate and dodecyl gallate, by contrast, are obtained by chemical synthesis. The gallates can trigger allergies in sensitive persons. Further usable antioxidants in compositions of the present invention are: sulfur dioxide, E 220 sodium sulfite, E 221 sodium hydrogensulfite, E 222 sodium disulfite, E 223 potassium disulfite, E 224 calcium sulfite, E 226 calcium hydrogensulfite, E 227 potassium hydrogensulfite, E 228 lactic acid, E 270 ascorbic acid, E 300 sodium L-ascorbate, E 301 calcium L-ascorbate, E 302 ascorbic esters, E 304 tocopherol, E 306 alpha-tocopherol, E 307 gamma-tocopherol, E 308 delta-tocopherol, E 309 propyl gallate, E 310 octyl gallate, E 311 dodecyl gallate, E 312 isoascorbic acid, E 315 sodium isoascorbate, E 316 tert-butylhydroquinone (TBHQ), E 319 butylhydroxianisole, E 320 butylhydroxytoluene, E 321 lecithin, E 322 citric acid, E 330 salts of citric acid (E 331 & E 332) sodium citrate, E 331 potassium citrate, E 332 calcium-disodium-EDTA, E 385 diphosphates, E 450 disodium diphosphate, E 450a trisodium diphosphate, E 450b tetrasodium diphosphate, E 450c dipotassium diphosphate, E 450d tripotassium diphosphate, E 450e dicalcium diphosphate, E 450f calcium dihydrogendiphosphate, E 450g triphosphates, E 451 pentasodium triphosphate, E 451a pentapotassium triphosphate, E 451b polyphosphate, E 452 sodium polyphosphate, E 452a potassium polyphosphate, E 452b sodium calcium polyphosphate, E 452c calcium polyphosphate, E 452d tin(II) chloride, E 512.

Vitamins have a wide variety of different mechanisms of biochemical action. Some act like hormones and regulate mineral metabolism (e.g. vitamin D), or act on the growth of cells and tissue and cell differentiation (e.g. some forms of vitamin A). Others are antioxidants (e.g. vitamin E and, under some circumstances, vitamin C as well). The majority of vitamins (e.g. the B vitamins) are precursors of enzymatic cofactors that assist enzymes in catalysing particular processes in metabolism. In this connection, vitamins may sometimes be tightly bound to the enzymes, for example as part of the prosthetic group: one example of this is biotin, which is part of the enzyme responsible for the formation of fatty acids. Vitamins may alternatively also be less strongly bound and in that case act as co-catalysts, for example as groups that can be eliminated readily and transport chemical groups or electrons between the molecules. For example, folic acid transports methyl, formyl and methylene groups into the cell. Even though their assistance in enzyme-substrate reactions is well known, the other properties thereof are also of great significance for the body.

In the context of the present invention, useful vitamins are substances selected from the group consisting of
vitamin A (retinol, retinal, betacarotene),
vitamin B₁ (thiamine),
vitamin B₂ (riboflavin),
vitamin B₃ (niacin, niacinamide),
vitamin B₅ (pantothenic acid),
vitamin B₆ (pyridoxine, pyridoxamine, paridoxal),
vitamin B₇ (biotin),
vitamin B₉ (folic acid),
vitamin B₁₂ (cyanocobalamin, hydroxycobalamin, methylcobalamin),
vitamin C (ascorbic acid),
vitamin D (cholecalciferol),
vitamin E (tocopherols, tocotrienols) and
vitamin K (phylloquinone, menaquinone).
   The preferred vitamins, aside from ascorbic acid, are the group of the tocopherols.

### Salts

Useful salts (component c13) include sodium chloride, magnesium chloride and potassium chloride.

### Food Dyes

Food dyes or dyes for short (component c14) are food additives for colouring foods. Dyes are divided into the groups of natural dyes and synthetic dyes. The nature-identical dyes are likewise of synthetic origin. The nature-identical dyes are synthetic analogues of naturally occurring colouring substances. Suitable dyes for use in the present composition are selected from: curcumin, E 100 riboflavin, lactoflavin, vitamin B2, E 101 tartrazine, E 102 quinoline yellow, E 104 yellow-orange S, yellow-orange RGL, E 110 cochineal, carminic acid, true carmine, E 120 azorubine, carmoisine, E 122 amaranth, E 123 cochineal red A, Ponceau 4 R, Victoria scarlet 4 R, E 124 erythrosine, E 127 Allura red AC, E 129 Patent blue V, E 131 indigotin, indigo carmine, E 132 Brilliant Blue FCF, Patent Blue AE, Amido Blue AE, E 133 chlorophylls, chlorophyllins, E 140 copper complexes of chlorophylls, copper-chlorophyllin complex, E 141 Brilliant Acid Green, Green S, E 142 caramel colour, E 150 a sulfite lye caramel colour, E 150 b ammonia caramel colour, E 150 c ammonium sulfite caramel colour, E 150 d Brilliant Black FCF, Brilliant Black PN, Black PN, E 151 vegetable charcoal, E 153 Brown FK, E 154 Brown HAT, E 155 carotene, E 160 a annatto, bixin, norbixin, E 160 b capsanthin, capsorubin, E 160 c lycopene, E 160 d beta-apo-8′-carotenal, apocarotenal, beta-apocarotenal, E 160 e beta-apo-8′-carotenoic acid ethyl ester (C30), apocarotene esters, beta-carotenoic esters, E 160 f lutein, xanthophyll, E 161 b canthaxanthin, E 161 g betanin, beet red, E 162 anthocyans, E 163 calcium carbonate, E 170 titanium dioxide, E 171 iron oxides, iron hydroxides, E 172 aluminium, E 173 silver, E 174 gold, E 175 lithol rubine BK, rubine pigment BK, E 180.

### Preferred Mixtures

In a preferred embodiment, the formulations may have the following composition, based in each case on the total weight:
(a) about 10% to about 95%, preferably about 50% to about 80%, by weight of milk proteins and/or plant proteins;
(b) about 5% to about 20% by weight, preferably about 10% to about 15% by weight, of oligosaccharides;
(c1) 0% to about 20%, preferably about 1% to about 10%, by weight, of animal and/or vegetable fats;
(c2) 0% to about 5% by weight, preferably about 1% to about 2% by weight, of starch products;
(c3) 0% to about 5% by weight, preferably about 1% to about 2% by weight, of dietary fibres;
(c4) 0% to about 5% by weight, preferably about 1% to about 2% by weight, of emulsifiers;
(c5) 0% to about 2% by weight, preferably about 0.5% to about 1% by weight, of thickeners;
(c6) 0% to about 2% by weight, preferably about 0.5% to about 1% by weight, of flavourings;
(c7) 0% to about 1% by weight, preferably about 0.1% to about 0.5% by weight, of probiotic microorganisms;
(c8) 0% to about 1% by weight, preferably about 0.1% to about 0.5% by weight, of probiotic substances;
(c9) 0% to about 1% by weight, preferably about 0.1% to about 0.5% by weight, of food acids,
(c10) 0% to about 1% by weight, preferably about 0.1% to about 0.5% by weight, of sweeteners;
(c11) 0% to about 1% by weight, preferably about 0.1% to 0.5% by weight, of antioxidants and/or vitamins;
(c12) 0% to about 1% by weight of rennet;
(c15) 0% to about 1% by weight of salts;
(c14) 0% to about 0.5% by weight of dyes;
   with the proviso that the stated amounts, optionally together with water, add up to 100% by weight. The further auxiliaries and additives are elucidated by way of example hereinafter. For the sake of good order, it should be pointed out that formulations that do not add up to 100% by weight in this way are not part of the invention, and it is possible for the person skilled in the art at any time, on the basis of the present description, to select formulations wherein the components add up to 100% by weight and satisfy the technical teaching.

The formulations may take the form of aqueous solutions or dispersions, but are preferably powders, specifically dry powders, meaning that they have a residual moisture content of less than 5% by weight.

In association with the forms of administration of the formulations, a process for producing the above-described prebiotic formulations is also claimed, comprising or consisting of the following steps:
(i) providing an aqueous solution of the protein component;
(ii) providing an aqueous solution of the oligosaccharide component;
(iii) mixing the two components; optionally
(iv) dewatering the mixture; and optionally
(v) mixing in the further additives.

Aside from freeze-drying—which will generally be too costly—spray-drying is the preferred method of dewatering. The concentrated and preheated solutions or dispersions comprising components (a) and (b) are sprayed by means of a tower at temperatures of about 80 to 95° C.

### INDUSTRIAL APPLICABILITY

The invention further relates to the use of the formulations firstly as food supplements and secondly for animal nutrition.

The invention further relates, finally, to the use of the oligosaccharides mentioned for masking the bitter taste of plant proteins. It is already sufficient here to add oligosaccharides in amounts of about 1% to about 5% by weight to the proteins.

### EXAMPLES

### Production Example 1

### Production of β-GOS with High DP Proceeding from Lactose Solution

1000 kg of a 30% by weight lactose solution was heated to 98° C., and sterilized, in a tubular heat exchanger for 120 seconds. The sterilized solution was cooled down to 55° C., transferred into a first fermenter, adjusted to a pH of 6.5 with the aid of lactic acid, admixed with beta-galactosidase from Bacillus circulans in an enzyme:substrate weight ratio of 1:500, and stirred. The progress of the transgalactosylation was monitored by sampling. After about 90 minutes, the maximum GOS concentration had been attained. The pH was increased to 10 by addition of 30% by weight sodium hydroxide solution within a few minutes, which abruptly reduced the activity of the enzyme by 80%. The reaction mixture was directed to an ultrafiltration unit provided with a spiral-wound membrane having a pore size of 10 kDa. The inactivated enzyme material was returned to the fermenter with the retentate R1; in order to compensate for loss, 5% by weight of fresh enzyme based on the starting amount was added.

The permeate P1 was transferred to a second fermenter and admixed with beta-galactosidase from Aspergillus oryzae in an enzyme:substrate weight ratio of 1:500, the pH was adjusted to the enzyme optimum and the mixture was stirred. The progress of the transgalactosylation was again monitored by sampling. After about 90 minutes, the maximum GOS concentration had been attained. The pH was adjusted to 10.0 by addition of 30% by weight sodium hydroxide solution within a few minutes, which abruptly reduced the activity of the enzyme by 80%.

The reaction mixture was directed to a second ultrafiltration unit likewise provided with a spiral-wound membrane having a pore size of 10 kDa. The inactivated enzyme material was returned to the fermenter with the retentate R2; in order to compensate for loss, 5% by weight of fresh enzyme based on the starting amount was added.

The permeate P2 was directed to a nanofiltration unit provided with a ceramic membrane having a pore size of 1000 Da. With permeate P3, the monosaccharides still present in the product were removed, while retentate R3 was fed to a reverse osmosis unit that worked with a concentration factor of 1:2. The permeate P3 obtained (i.e. the concentration water) was returned to the process; the retentate R3 (i.e. the GOS concentrate) was heated to about 85° C. in a plate heat exchanger for 30 seconds and sprayed by means of a tower. A white powder having a GOS content of more than 75% by weight was obtained, which still had a residual moisture content of 1% by weight. The average DP of the GOS was 3-4.

### Production Example 2

### Production of GOS by the Neutral Method Proceeding from Sucrose Solution

1000 kg of a 30% by weight sucrose solution was heated to 98° C., and sterilized, in a tubular heat exchanger for 120 seconds. The sterilized solution was cooled down to 55° C., transferred into a fermenter, adjusted to a pH of 4.5 with the aid of lactic acid, admixed with alpha-galactosidase from Aspergillus niger in an enzyme:substrate weight ratio of 1:50, and stirred. The progress of the transgalactosylation was monitored by sampling. After about 90 minutes, the maximum GOS concentration had been attained. The pH was increased to 10 by addition of 30% by weight sodium hydroxide solution within a few minutes, which abruptly reduced the activity of the enzyme by 80%. The reaction mixture was directed to an ultrafiltration unit provided with a spiral-wound membrane having a pore size of 10 kDa. The inactivated enzyme material was returned to the fermenter with the retentate R1; in order to compensate for loss, 5% by weight of fresh enzyme based on the starting amount was added. The permeate P1 was directed to a nanofiltration unit provided with a ceramic membrane having a pore size of 1000 Da. With permeate P2, the monosaccharides still present in the product were removed, while retentate R2 was fed to a reverse osmosis unit that worked with a concentration factor of 1:2. The permeate P3 obtained (i.e. the concentration water) was returned to the process; the retentate R3 (i.e. the GOS concentrate) was heated to about 85° C. in a plate heat exchanger for 30 seconds and sprayed by means of a tower. A white powder having a GOS content of more than 75% by weight was obtained, which still had a residual moisture content of 1% by weight. The DP of the GOS was about 5.

### Production Example 3

Such an amount of proteins was added to the aqueous oligosaccharide concentrates from production examples 1 and 2 as to result in a weight ratio of oligosaccharide to proteins of 20:80. Subsequently, the mixtures were diluted to a solids concentration of 60% by weight, heated to about 85° C. in a plate heat exchanger for 30 seconds, and sprayed by means of a tower. A white powder was obtained, which still had a residual moisture content of 2.2% by weight.

### Formulation Examples

GOS according to Production Example 1 was added to aqueous solutions of various proteins in different amounts and assessed by 5 testers with regard to their taste qualities according to the following scheme:
(1)=unpleasantly bitter
(2)=bitter
(3)=neutral
(4)=sweetish
(5)=unpleasantly sweet
   The standard used was solutions of the proteins without addition of oligosaccharides and protein solutions that contained lactose rather than GOS. The results are summarized in Table 1; Examples 1 to 6 are inventive; Examples V1 and V2 serve for comparison.

TABLE 1
Taste assessment

| Components | 1 | 2 | 3 | 4 | 5 | 6 | V1 | V2 |
|---|---|---|---|---|---|---|---|---|
| Potato protein | 5.0 | 5.0 | 5.0 | — | — | — | — | — |
| Lupin protein | — | — | — | 5.0 | 5.0 | 5.0 | 5.0 | 5.0 |
| B-GOS | 0.1 | 0.5 | 1.0 | 0.1 | 0.5 | 1.0 | — | — |
| Lactose | — | — | — | — | — | — | — | 1.0 |

| | |
|---|---|
| Water | to 100 |

Taste assessment

| | | | | | | | |
|---|---|---|---|---|---|---|---|
| 2 | 3 | 4 | 2 | 3 | 4 | 1 | 2 |

### The Examples and Comparative Examples Show:

GOS reduce the bitter taste of plant proteins even in small concentrations of 1:50;
Even in high concentrations as are typical in the formulations of the invention, the addition of GOS does not lead to unpleasant sweetness, but only to a sweetish taste;
Lactose even in a use ratio of 1:20 is incapable of covering up the bitter taste of the plant proteins.

What is claimed is:

1. A prebiotic formulation comprising or consisting of
a) milk proteins and/or plant proteins and
b) oligosaccharides selected from beta-galactooligosaccharides (β-GOS), alpha-galactooligosaccharides (α-GOS), fructooligosaccharides (FOS) and/or mannanoligosaccharides (MOS).

2. The formulation of claim 1, wherein the milk proteins are selected from the group formed by casein, milk protein concentrates, milk protein isolates, whey proteins, whey protein concentrates and protein-containing milk powders.

3. The formulation of claim 1, wherein the plant proteins are selected from the group formed by potato proteins, soya proteins, pea proteins, lupin proteins, oilseed rape proteins, sunflower proteins, pumpkinseed proteins and mixtures thereof.

4. The formulation of claim 1, wherein the beta-galactooligosaccharides are obtained by transgalactosylation of lactose in the presence of beta-galactosidases.

5. The formulation of claim 1, wherein the alpha-galactooligosaccharides are obtained by transgalactosylation of sucrose in the presence of alpha-galactosidases.

6. The formulation of claim 1, wherein the fructooligosaccharides are obtained by enzymatic processes or hydrolysis of inulin.

7. The formulation of claim 1, wherein the mannanoligosaccharides are obtained from yeast cells.

8. The formulation of claim 1, wherein the oligosaccharides have a DP in the range from about 5 to about 80.

9. The formulation of claim 1, wherein further comprising at least one additive selected from the group formed by starch products, dietary fibres, thickeners, flavourings, further prebiotics, probiotics, food acids, salts and dyes, and mixtures thereof.

10. The formulation of claim 1, wherein the formulation has the following composition:
(a) about 10% to about 95% by weight of milk proteins and/or plant proteins;
(b) about 5% to about 20% by weight of oligosaccharides;
(c1) 0% to about 5% by weight of animal fats and/or vegetable fats;
(c2) 0% to about 5% by weight of starch products;
(c3) 0% to about 5% by weight of dietary fibers;
(c4) 0% to about 5% by weight of emulsifiers;
(c5) 0% to about 2% by weight of thickeners;
(c6) 0% to about 2% by weight of flavourings;
(c7) 0% to about 1% by weight of probiotic microorganisms;
(c8) 0% to about 1% by weight of prebiotic substances;
(c9) 0% to about 1% by weight of food acids;
(c10) 0% to about 1% by weight of sweeteners;
(c11) 0% to about 1% by weight of antioxidants and/or vitamins;
(c12) 0% to about 1% by weight of rennet;
(c13) 0% to about 1% by weight of salts;
(c14) 0% to about 0.5% by weight of dyes;
with the proviso that the stated amounts, optionally together with water, add up to 100% by weight.

11. The formulation of claim 1, wherein the formulation is in the form of an aqueous solution or dispersion or powder.

12. Process for producing a prebiotic formulation according to claim 1, comprising or consisting of the following steps:
(i) providing an aqueous solution of the protein component;
(ii) providing an aqueous solution of the oligosaccharide component;
(iii) mixing the two components; optionally
(iv) dewatering the mixture; and optionally
(v) mixing in the further additives.

13. The formulation of claim 1 comprised in a food supplement.

14. The formulation of claim 1 comprised in an animal nutrition composition.

15. A method of masking bitterness of a plant protein, the method comprising mixing one or more oligosaccharides selected from the group formed by beta-galactooligosaccharides (β-GOS), alpha-galactooligosaccharides (α-GOS), fructooligosaccharides (FOS) and mannanoligosaccharides (MOS) in a composition with the plant protein.
